(19) United States
(12) Patent Application Publication
(10) Pub. No.: US 2001/0007766 A1
(43) Pub. Date: Jul. 12, 2001

(54) Novel method to sterilize recombinant products

(76) Inventors: Alan I. Rubinstein, Los Angeles, CA (US); Daniel P. Rubinstein, Brookline, MA (US)

Correspondence Address: ST. ONGE STEWARD JOHNSTON & REENS, LLC, 986 BEDFORD STREET, STAMFORD, CT, 06905-5619, US

(21) Appl. No.: 09/795,738

(22) Filed: Feb. 28, 2001

Related U.S. Application Data
(63) Continuation of application No. 08/184,308, filed on Jan. 21, 1994, now abandoned.

(51) Int. Cl. C12N 7/04

(52) U.S. Cl. 435/236

(57) ABSTRACT

Recombinant proteins, manufactured with genetic engineering techniques, like its counterpart human derived proteins, for example, recombinant Factor VIII have the potential for transmission of viruses and bacteria which are used in the process. This invention demonstrates that one may dry heat at high temperature, the lyophilized final Factor VIII product even at high temperatures and still show significant VIII:C activity. Such terminal dry heat will assure that there has been recontamination.

### PREVIOUS APPLICATION

This is a continuation of U.S. patent application No. 08/184,308, filed 01/21/94 which is a continuation of a U.S. patent application filed in February 1993.

### INTRODUCTION

Recombinant proteins, manufactured with genetic engineering techniques, like its counterpart human derived proteins, for example, recombinant Factor VIII have the potential for transmission of viruses and bacteria which are used in the process. This invention demonstrates that one may dry heat at high temperatures, the lyophilized final Factor VIII product even at high temperatures and still slow significant VIII:C activity. Such terminal dry heat will assure that there has not been recontamination.

### DISCLOSURE

There are several steps and processes in the manufacture of a recombinant product, for instance, recombinant Factor VIII where there can be concerns of contamination and where an inexpensive sterilization step would be of great benefit. For instance: Chinese Hamster Ovary (CHO) cells are the cell line. The master cell bank (MCB) is established by taking cells adapted to suspension culture. CHO cells are tumorigenic and there still remain residual DNA, which has been reported as less than 10 pg DNA, as well as residual CHO protein which has been measured as less than 20 Mgr (Transfusion Medicine Reviews, Vol. VI, No. 4 [1972], p. 250). It would be desirable to denature the residual DNA and this is thus another advantage of this invention, high temperature terminal dry heat may also destroy or denature DNA and render any residual nucleic acid (i.e., DNA or RNA) non functional. The cell lines themselves may also have viral contamination as well as the monochonal antibody may be contaminated with virus deposits procedures designed to minimize this.

Steps to ensure viral safety have been introduced at all phases of the production cycle, including validation of the cell lines as virus free, evaluation and documentation of raw materials, and MoAb as virus free, and the testing of material at various phases of manufacturing for the presence of any possible adventitious virus contaminants.

Table 1 demonstrates minimal loss of VIII:C following heating of lyophilized Factor VIII (recombinant Baxter-Hyland and Genetics Institute) at 70° C. for 2 hours and at 100° C. (in boiling water) for 7½minutes. The procedure was as follows:

Samples: Baxter Hyland® Antihemophilic Factor (Recombinant) Recombinate™ Lot #2938X003AA.

The vial from the bottle heated in boiling water for 7½was reconstituted with the 10 ml of sterile water provided with the product. The vial went into solution readily. Each vial was then diluted to 1 lU/ml. Each vial was marked as containing 251 lU/bottle or 251 IU/10 ml which equals 25.1 lU/ml. A 1:25 dilution was made in the assay buffer to yield a concentration of 1 lU/ml. This initial 1:25 dilution was then assayed for VIII:C at dilutions of 1:10, 1:20, and 1:40. Example 2 further demonstrates recovery after dry heating at 80° for 10 hours and 100° C. for 7.5 minutes.

### EXAMPLE 1

### HEATED LYOPHILIZED RECOMBINANT FACTOR VII CONCENTRATE

| | | | Heated at 100° C. |
|---|---|---|---|
| | | Heated at 70° C. | (in boiling water |
| Dilution | Control | for approx. 2 hours | for 7½ minutes) |
| 1:10 | 34% | 37% | 43% |
| 1:20 | 34% | 35% | 46% |
| 1:40 | 32% | 33% | 44% |

### EXAMPLE 2

### HEATED LYOPHILIZED RECOMBINANT FACTOR VII CONCENTRATE

Comparison of antihemophilic factor recombinant dry heat treated at different conditions:

1. Bottle A: Control

2. Bottle B: Immersed in 80° C. water bath for 10 hours

3. Bottle C: Immersed in 100° C. boiling water for 7.5 minutes

Antihemophilic factor (recombinant)

Lot #2938X004AE

AHF 234 lU/Bottle

Method of Assay: Tilt tube

Reference Control Plasma: Pacific Hemostasis UCRP (301T02)

Control Reference Plasma: Pacific Hemostasis ACRP (603R02)

APTT Reagent: Organon Teknika Automatic APTT (102101)

Each bottle was reconstituted according to manufacture recommendation after treatment. A 1:100 dilution was made of each bottle. From each dilution a 1:20 and 1:40 was prepared and the factor VIII activity was determined.

Control Bottle (untreated): 205 lU/bottle

Bottle at 80° C./10 hours: 131 lU/bottle

Bottle at 100° C./7.5 minutes: 182 lU/bottle

Bottle at 80° C. lost 36% activity

Bottle at 100° C. lost 11% activity

Solubility was grossly unremarkable as compared to control

Other temperatures which can be used are 60° C. to over 100° C., however, 80° C. to 100° C. are preferred. It is preferred that the bottle of lyophilized material be placed in a heated water bath, since dry heating in an oven takes more time to penetrate the lyophilized material. Also additional diluent may be added to increase solubility.

The same can be applied to other recombinant products, for instance: interleukemia, Growth Hormones, Factor IX and others (Biotech 9:1344-1355, 1991 and M. Cell Bio. 9:1233-1239, 1989).

It is thus apparent to those skilled in the art that various modifications in time and temperature and purity and composition of the material being heated may be made by those skilled in the art without departing from the scope and spirit of this invention which is to be bounded by the following:

What is claimed is:

1. A method to inactivate any microorganism or virus in a genetic manufactured product by lyophilizing and then heating at a predetermined temperature and time at a predetermined pressure until there is significant microbiologic or viral inactivation.

2. A method as in claim 1 where the genetic engineered product is a product manufactured through recombinant technology.

3. A method as in claim 1 where the product is recombinant Factor VIII.

4. A method as in claim 1 whereby the heating temperature is approximately 60° C. to 100° C., said time of heating decreasing with increasing temperature.

5. A method as in claim 1 wherein the temperature of heating is 100° C. and the time of heating is approximately 7½minutes to ½hour.

6. A method as in claim 1 wherein the temperature of heating is 80° C.
